(19) United States
(12) Patent Application Publication
(10) Pub. No.: US 2024/0000394 A1
(43) Pub. Date: Jan. 4, 2024

(54) Deep Learning-Based Wearable Electro-Tonoarteriography (ETAG) Processing Method And Apparatus For Estimation of Continuous Arterial Blood Pressure

(71) Applicant: Hong Kong Centre for Cerebro-Cardiovasculer Health Engineering Limited, Hong Kong (CN)

(72) Inventors: Yuanting ZHANG, Hong Kong (CN); Ting XIANG, Hong Kong (CN); Nan JI, Hong Kong (CN); David A. CLIFTON, Oxford (GB); Lei LU, Oxford (GB); Tingting ZHU, Oxford (GB)

(21) Appl. No.: 17/981,391

(22) Filed: Nov. 5, 2022

(30) Foreign Application Priority Data
Jun. 29, 2022 (CN) 202210760467.0

(51) Int. Cl. A61B 5/00; A61B 5/282; A61B 5/256; A61B 5/346; A61B 5/332; G16H 50/20; G16H 40/67

(52) U.S. Cl. CPC A61B 5/7267; A61B 5/282; A61B 5/256; G16H 40/67; A61B 5/332; A61B 5/0006; G16H 50/20; A61B 5/346

(57) ABSTRACT

The present invention provides a deep learning-based wearable electro-tonoarteriography method and apparatus for the estimation of continuous arterial blood pressure, which relates to the technical fields of medical detection and artificial intelligence, and is applicable to, such as, tonoarteriogram (TAG, which is continuous arterial blood pressure) signal estimation and cardiac diseases detection. The method comprises: acquiring at least one lead ECG signal collected from a clothing and/or a wearable device worn by a subject of detection; processing the ECG signal based on a deep learning network, determining a signal processing result related to a tonoarteriogram information and/or related to a cardiac disease information. The present invention is advantageous in realizing the acquisition of continuous arterial blood pressure signal and/or the automatic diagnosis of cardiac disease on the basis of ensuring accuracy.

### FIELD OF THE INVENTION

The present invention relates to the technical field of medical testing and artificial intelligence, specifically, this invention relates to a deep learning-based wearable electro-tonoarteriography (ETAG) processing method and apparatus for the estimation of continuous arterial blood pressure.

### BACKGROUND OF THE INVENTION

According to the World Health Organization (WHO), cardiovascular disease (CVD) is the leading cause of death and disability worldwide, accounting for approximately one-third of all deaths in the world. Among the multiple risk factors for CVD, hypertension is at the top of the list, making continuous non-invasive blood pressure monitoring in daily life very important. In addition, more than two-thirds of sudden cardiac deaths occur outside of hospitals. ECG is commonly used to diagnose and monitor abnormal heart conditions such as atrial fibrillation, ventricular fibrillation, and myocardial infarction, and is the most common method to screen and detect heart disease. Therefore, there is an urgent need to use wearable devices to accurately and unobtrusively monitor individual cardiovascular disease risk factors in daily life.

Currently, there are some wearable devices available in the market for ECG signal detection, however, the function of this device is mainly for heart rate, heart rate variability and arrhythmia detection, but not for obtaining blood pressure and its blood pressure related information through ECG signal. Currently, there have been some studies on the application of machine learning to obtain blood pressure, but it is not possible to obtain tonoarteriogram (TAG) signal, which is a continuous arterial blood pressure signal with more physiological significance and blood pressure information; or it still requires the combination of ECG signal and photoplethysmographic signal to obtain blood pressure signal.

### OBJECTS OF THE INVENTION

An object of the present invention is to provide a a deep learning-based wearable electro-tonoarteriography (ETAG) processing method and apparatus for the estimation of continuous arterial blood pressure.

The above object is met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

Embodiments of the present application provide a deep learning-based wearable electro-tonoarteriography (ETAG) processing method and apparatus for the estimation of continuous arterial blood pressure to solve at least one of the above technical problems.

In a first aspect, embodiments of the present application provide a deep learning-based wearable electro-tonoarteriography (ETAG) processing method for the estimation of continuous arterial blood pressure, comprising: acquiring at least one lead ECG signal collected from a clothing and/or a wearable device worn by a subject of detection; processing the ECG signal(s) based on a deep learning network, determining a signal processing result related to a tonoarteriogram information and/or related to a cardiac disease information.

In a possible embodiment, the said wearable device is integrated with an ECG electrode; the step of acquiring the at least one lead ECG signal collected from a clothing and/or a wearable device worn by a subject of detection comprises at least one of the following: acquiring a lead ECG signal I of a limb collected when the wearable device is worn at a right hand and the ECG electrode of the wearable device is in contact with a left hand of the subject of detection; acquiring a lead ECG signal II of a limb collected when the wearable device is worn at a right hand and the wearable device is in contact with a left side of a neck or a region above the left side of the neck of the subject of detection; acquiring a lead ECG signal I, II or III collected when the wearable device worn at a right hand and the ECG electrode of the wearable device is in contact with a left hand and a left side of a neck, or a region above the left side of the neck of the subject of detection; acquiring at least one lead from twelve-lead ECG signals collected from a first clothing worn by the subject of detection; the first clothing comprises the ECG electrode separately arranged at a chest portion, a wrist and an ankle; acquiring at least one lead from a fifteen-lead ECG signals collected from a second clothing worn by the subject of detection; the second clothing comprises the ECG electrode separately arranged on a chest portion, a back, a wrist and an ankle; acquiring a lead ECG signal VI of a chest portion collected from a third clothing worn by the subject of detection; the third clothing comprises an ECG electrode arranged at the chest portion.

In a possible embodiment, the said ECG electrode comprises at least one of a dry electrode, a wet electrode, a flexible electrode, a hydrogel ion electrode, an electronic fabric electrode and contactless electronics.

In a possible embodiment, the said step of processing the ECG signal based on the deep learning network, determining the signal processing result related to the tonoarteriogram information and/or related to the cardiac disease information comprises: acquiring a low-frequency signal related to blood pressure from the ECG signal; processing the ECG signal and the low-frequency signal by the deep learning network, determining the signal processing result related to the tonoarteriogram information and/or related to the cardiac disease information.

In a possible embodiment, the said step of processing the ECG signal and the low-frequency signal by the deep learning network, determining the signal processing result related to the tonoarteriogram information and/or related to the cardiac disease information comprises: performing a feature extraction on the ECG signal and the low-frequency signal by a frequency-domain based deep neural network to obtain a frequency domain feature information, and performing a feature extraction on the ECG signal and the low-frequency signal by a time-domain based deep neural network to obtain a time domain feature information; performing a pooling operation on the frequency domain feature information and the time domain feature information to obtain a pooled feature information; performing a feature fusion classification processing against the pooled feature information by a fully connected network, determining the signal processing result related to the tonoarteriogram information and/or related to the cardiac disease information.

In a possible embodiment, the said time-domain based deep neural network comprises a sequentially connected convolutional neural network and at least one layer of long short-term memory neural network; the step of performing the feature extraction on the ECG signal and the low-frequency signal by the time-domain based deep neural network to obtain the time domain feature information comprises: performing the feature extraction on the ECG signal and the low-frequency signal by the convolutional neural network to obtain a convolutional feature information; processing the convolutional feature information by the long short-term memory neural network to obtain the time domain feature information; the time domain feature information comprises dependent information of blood pressure dynamic to time.

In a possible embodiment, the said time-domain based deep neural network is a time-domain based interpretable deep learning network; steps of constructing the network comprises: re-structuring the input ECG signal and the low-frequency signal by an input layer; extracting a re-structured feature information of the ECG signal and the low-frequency signal by a convolutional layer; performing a pooling process on the feature information obtained from convolution by a pool layer; processing, sequentially, the feature information obtained from pooling by a previous pooling layer by adding any number of the convolutional layer and the pooling layer; performing, by a fully connected layer, a classification on the feature information obtained from the pooling by a last pooling layer, the fully connected layer is provided with a regularization parameter; calculating a deviation of a result of the classification and evaluating accuracy of a current network.

In a possible embodiment, the said signal processing result related to the tonoarteriogram information comprises at least one of a tonoarteriogram signal, a systolic blood pressure information, a diastolic blood pressure information, a blood pressure variation information and a high blood pressure information; the signal processing result related to the cardiac disease information comprises at least one of an electrocardiogram, an arrhythmia test result and a myocardial infarction test result.

In a possible embodiment, further comprising at least one of the following: transmitting the signal processing result to a user apparatus to display the signal processing result to a user; the user apparatus comprises at least one of a mobile phone, a watch and glasses; uploading the signal processing result to a cloud database and/or a medical platform.

In a second aspect, embodiments of the present application provide a deep learning-based wearable electro-tonoarteriography (ETAG) apparatus for the estimation of continuous arterial blood pressure, comprising: an acquiring unit for acquiring at least one lead ECG signal collected from a clothing and/or a wearable device worn by a subject of detection; a processing unit for processing the ECG signal based on a deep learning network, determining a signal processing result related to a tonoarteriogram information and/or related to a cardiac disease information.

In a possible embodiment, the said wearable device is integrated with an ECG electrode; the step of acquiring the at least one lead ECG signal collected from a clothing and/or a wearable device worn by a subject of detection comprises at least one of the following: acquiring a lead ECG signal I of a limb collected when the wearable device is worn at a right hand and the ECG electrode of the wearable device is in contact with a left hand of the subject of detection; acquiring a lead ECG signal II of a limb collected when the wearable device is worn at a right hand and the wearable device is in contact with a left side of a neck or a region above the left side of the neck of the subject of detection; acquiring a lead ECG signal I, II or III collected when the wearable device worn at a right hand and the ECG electrode of the wearable device is in contact with a left hand and a left side of a neck, or a region above the left side of the neck of the subject of detection; acquiring at least one lead from twelve-lead ECG signals collected from a first clothing worn by the subject of detection; the first clothing comprises the ECG electrode separately arranged at a chest portion, a wrist and an ankle; acquiring at least one lead from a fifteen-lead ECG signals collected from a second clothing worn by the subject of detection; the second clothing comprises the ECG electrode separately arranged on a chest portion, a back, a wrist and an ankle; acquiring a lead ECG signal VI of a chest portion collected from a third clothing worn by the subject of detection; the third clothing comprises an ECG electrode arranged at the chest portion.

In a possible embodiment, the said ECG electrode comprises at least one of a dry electrode, a wet electrode, a flexible electrode, a hydrogel ion electrode, an electronic fabric electrode and contactless electronics.

In a possible embodiment, the said step of processing the ECG signal based on the deep learning network, determining the signal processing result related to the tonoarteriogram information and/or related to the cardiac disease information comprises: acquiring a low-frequency signal related to blood pressure from the ECG signal; processing the ECG signal and the low-frequency signal by the deep learning network, determining the signal processing result related to the tonoarteriogram information and/or related to the cardiac disease information.

In a possible embodiment, the said step of processing the ECG signal and the low-frequency signal by the deep learning network, determining the signal processing result related to the tonoarteriogram information and/or related to the cardiac disease information comprises: performing a feature extraction on the ECG signal and the low-frequency signal by a frequency-domain based deep neural network to obtain a frequency domain feature information, and performing a feature extraction on the ECG signal and the low-frequency signal by a time-domain based deep neural network to obtain a time domain feature information; performing a pooling operation on the frequency domain feature information and the time domain feature information to obtain a pooled feature information; performing a feature fusion classification processing against the pooled feature information by a fully connected network, determining the signal processing result related to the tonoarteriogram information and/or related to the cardiac disease information.

In a possible embodiment, the said time-domain based deep neural network comprises a sequentially connected convolutional neural network and at least one layer of long short-term memory neural network; the step of performing the feature extraction on the ECG signal and the low-frequency signal by the time-domain based deep neural network to obtain the time domain feature information comprises: performing the feature extraction on the ECG signal and the low-frequency signal by the convolutional neural network to obtain a convolutional feature information; processing the convolutional feature information by the long short-term memory neural network to obtain the time domain feature information; the time domain feature information comprises dependent information of blood pressure dynamic to time.

In a possible embodiment, the said time-domain based deep neural network is a time-domain based interpretable deep learning network; steps of constructing the network comprises: re-structuring the input ECG signal and the low-frequency signal by an input layer; extracting a re-structured feature information of the ECG signal and the low-frequency signal by a convolutional layer; performing a pooling process on the feature information obtained from convolution by a pool layer; processing, sequentially, the feature information obtained from pooling by a previous pooling layer by adding any number of the convolutional layer and the pooling layer; performing, by a fully connected layer, a classification on the feature information obtained from the pooling by a last pooling layer, the fully connected layer is provided with a regularization parameter; calculating a deviation of a result of the classification and evaluating accuracy of a current network.

In a possible embodiment, the said signal processing result related to the tonoarteriogram information comprises at least one of a tonoarteriogram signal, a systolic blood pressure information, a diastolic blood pressure information, a blood pressure variation information and a high blood pressure information; the signal processing result related to the cardiac disease information comprises at least one of an electrocardiogram, an arrhythmia test result and a myocardial infarction test result.

In a possible embodiment, the deep learning-based wearable electro-tonoarteriography (ETAG) apparatus for the estimation of continuous arterial blood pressure further comprises at least one of the following units: transmission unit, transmitting the signal processing result to a user apparatus to display the signal processing result to a user; the user apparatus comprises at least one of a mobile phone, a watch and glasses; upload unit, uploading the signal processing result to a cloud database and/or a medical platform.

In a third aspect, embodiments of the present application provide an electronic apparatus, the said electronic apparatus, comprising: a memory, a processor and a computer program stored in the memory, wherein the processor executes the computer program to implement the method steps of any one of the deep learning-based wearable electro-tonoarteriography (ETAG) processing method for the estimation of continuous arterial blood pressure provided in the first aspect above.

In a fourth aspect, embodiments of the present application provide a computer-readable storage medium on which a computer program is stored, wherein, when the computer program is executed by a processor, implements the method steps of any one of the deep learning-based wearable electro-tonoarteriography (ETAG) processing method for the estimation of continuous arterial blood pressure provided in the first aspect above.

In a fifth aspect, embodiments of the present application provide a computer program product, comprising a computer program, wherein, when the computer program is executed by a processor, implements the method steps of any one of the deep learning-based wearable electro-tonoarteriography (ETAG) processing method for the estimation of continuous arterial blood pressure provided in the first aspect above.

In a sixth aspect, embodiments of the present application provide a system for a deep learning-based electro-tonoarteriography for the estimation of continuous arterial blood pressure, comprising an electronic apparatus and a clothing and/or a wearable apparatus for a subject of detection; the clothing and/or the wearable device collects at least one lead ECG signal of the subject of detection, and transmits the collected ECG signal to the electronic apparatus via a wireless unit; the electronic apparatus implements the method steps of any one of the deep learning-based wearable electro-tonoarteriography (ETAG) processing method for the estimation of continuous arterial blood pressure provided in the first aspect above.

Embodiments of the present application provide a deep learning-based wearable electro-tonoarteriography (ETAG) processing method and apparatus for the estimation of continuous arterial blood pressure; specifically, the acquisition of cardiac signals can be performed through clothing and/or wearable devices worn by the test subject, and since the clothing and wearable devices are very easily wearable by the test subject based on the testing needs, continuous signal acquisition can be performed, from which more physiologically meaningful signals for processing, the implementation of this operation facilitates the acquisition of continuous arterial blood pressure signals and/or automatic diagnosis of heart disease; on this basis, after acquiring the ECG signal in at least one lead, the ECG signal can be processed based on a deep learning network to determine the signal processing results related to tonoarteriogram information and/or heart disease information; the implementation of this operation can be directly based on the acquired ECG heart blood number for estimation of tonoarteriogram signal and heart disease, without the need to combine the ECG signal with the PPG signal for processing, which helps to reduce the cost of device fabrication and improve the accuracy of signal processing results.

### BRIEF DESCRIPTION OF THE DRAWINGS

FIG. 1 shows a schematic diagram of the deep learning-based wearable electro-tonoarteriography (ETAG) method;

FIG. 2 shows a structure diagram of the proposed ETAG system;

FIG. 3 shows another structure diagram of the proposed ETAG system;

FIG. 4a shows the acquisition of ECG signals of limb lead I;

FIG. 4b shows the acquisition of ECG signals of limb leads I, II and III;

FIG. 5a shows a different configuration of 15-lead ECG electrodes in accordance with an embodiment of the invention;

FIG. 5b shows a different configuration of 15-lead ECG electrodes in accordance with an embodiment of the invention;

FIG. 5c shows a different configuration of 15-lead ECG electrodes in accordance with an embodiment of the invention;

FIG. 6 shows an overall framework of the proposed processing model in accordance with an embodiment of the invention;

FIG. 7 shows a schematic diagram of a time domain-based deep neural network;

FIG. 8 shows another schematic diagram of a time domain-based deep neural network;

FIG. 9 shows a structure diagram of the proposed ETAG apparatus; and

FIG. 10 shows a schematic diagram of a structure of an electronic device.

### DESCRIPTION OF PREFERRED EMBODIMENTS

Embodiments of the present application are described below in connection with the accompanying drawings in the present application. It should be understood that the embodiments set forth below in connection with the accompanying drawings are exemplary descriptions for the purpose of explaining the technical solutions of the embodiments of the present application and do not constitute a limitation of the technical solutions of the embodiments of the present application.

It will be understood by those skilled in the art that, unless specifically stated, the singular forms “one”, “a”, “said” and “the” used herein “may also include the plural form. It should be further understood that the terms “includes” and “comprises” as used in the embodiments of the present application mean that the corresponding features may be implemented as the features, information, data, steps, operations, components and/or assemblies presented, but do not exclude the implementation of other features, information, data, operations, components and/or assemblies that are supported in the art. It should be understood that when we refer to a component being “connected” or “coupled” to another component, the component may be directly connected or coupled to the other component, or it may refer to the component and the other component being connected through an intermediate component. In addition, the “connection” or “coupling” as used herein may include wireless connection or wireless coupling. The term “and/or” as used herein indicates at least one of the items defined by the term, e.g., “A and/or B” may be implemented as “A or B” or “A and B”.

In order to make the purpose, technical solutions and advantages of the present application clearer, the present application embodiments are described in further detail below in conjunction with the accompanying drawings.

This application embodiment relates to Artificial Intelligence (AI) is a theory, method, technology, and application system that uses a digital computer or a machine controlled by a digital computer to simulate, extend, and expand human intelligence, perceive the environment, acquire knowledge, and use the knowledge to obtain optimal results. In other words, artificial intelligence is an integrated technology of computer science that attempts to understand the essence of intelligence and to produce a new intelligent machine that can respond in a similar way to human intelligence. Artificial intelligence is also the study of the design principles and implementation methods of various intelligent machines to make them capable of perception, reasoning and decision making. Artificial intelligence technology is a comprehensive discipline that covers a wide range of fields, both at the hardware and software levels. Basic AI technologies generally include technologies such as sensors, special AI chips, cloud computing, distributed storage, big data processing technologies, operation/interaction systems, mechatronics, etc. Artificial intelligence software technologies mainly include computer vision technology, speech processing technology, natural language processing technology, and machine learning/deep learning, autonomous driving, intelligent transportation, and several other major directions.

The deep learning-based wearable electro-tonoarteriography (ETAG) processing method for the estimation of continuous arterial blood pressure proposed in this application embodiment specifically involves Machine Learning (ML), which is a multi-disciplinary interdisciplinary discipline involving probability theory, statistics, approximation theory, convex analysis, algorithmic complexity theory and other disciplines. It specializes in studying how computers can simulate or implement human learning behaviors to acquire new knowledge or skills and reorganize existing knowledge structures to continuously improve their performance. Machine learning is the core of artificial intelligence and is the fundamental way to make computers intelligent, and its applications span all areas of artificial intelligence. Machine learning and deep learning usually include techniques such as artificial neural networks, confidence networks, reinforcement learning, transfer learning, inductive learning, and style teaching learning. For example, the tonoarteriogram signal can be estimated by a machine learning based model for the acquired signal, etc.

The deep learning-based wearable electro-tonoarteriography (ETAG) processing method for the estimation of continuous arterial blood pressure proposed in this application embodiment can be applied to scenarios such astonoarteriogram signal estimation, heart disease detection, etc.

The technical solutions of the present application embodiments and the technical effects resulting from the technical solutions of the present application are described below through the description of several exemplary embodiments. It should be noted that the following embodiments can be cross-referenced, borrowed or combined with each other, and the description of the same terms, similar features and similar implementation steps, etc. in different embodiments will not be repeated.

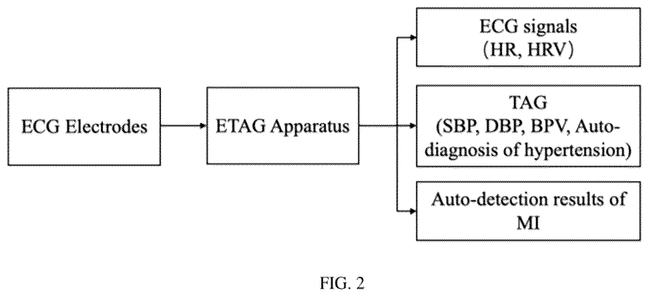

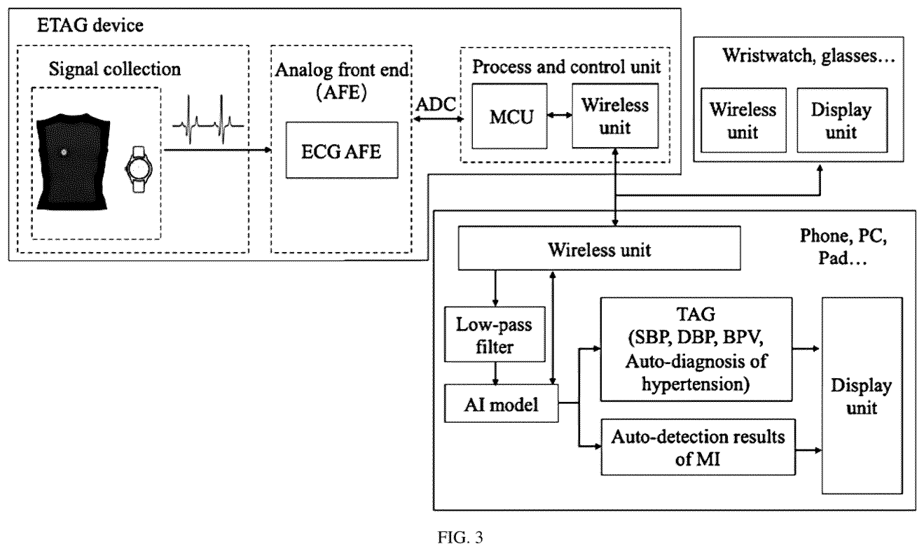

The present application provides a deep learning-based wearable electro-tonoarteriography (ETAG) processing method for the estimation of continuous arterial blood pressure, which, in its operating environment, may include a user terminal. As shown in FIGS. 2 and 3, the implementation of the method may first acquire the ECG signal acquired through the ECG electrodes designed in the ETAG device, and then process the acquired ECG signal through the user terminal to output the signal processing results related to the tonoarteriogram information and/or related to the heart disease information.

Wherein, the user terminal may run a client or a service platform. The terminal (which may also be referred to as a device) may be a smartphone, a tablet, a laptop, a desktop computer, a smart voice interaction device (e.g., a smart speaker), a wearable electronic device (e.g., a smart watch), a vehicle terminal, a smart home appliance (e.g., a smart TV), an AR/VR device, and the like, but is not limited thereto. In one example, the user terminal may be configured with an AI model for processing the ECG signal captured by the ETAG device.

In a possible embodiment, the operating environment of the present application may also include a server, such as transmitting the ECG signals collected by the ETAG device to the server for signal processing. The server may be a stand-alone physical server, a server cluster consisting of multiple physical servers or a distributed system (e.g., a distributed cloud storage system), or a cloud server providing cloud computing services.

In a possible embodiment, the terminal as well as the server can be connected directly or indirectly by wired or wireless communication, and the present application is not limited herein. For example, the terminal may send data acquisition requests to the server via the network.

In a possible embodiment, the operating environment may also include a database, and the database may be used to store data such as tonoarteriogram signals, heart disease information, etc.

The following is a specific description of the deep learning-based wearable electro-tonoarteriography (ETAG) processing method for the estimation of continuous arterial blood pressure provided in embodiments of the present application.

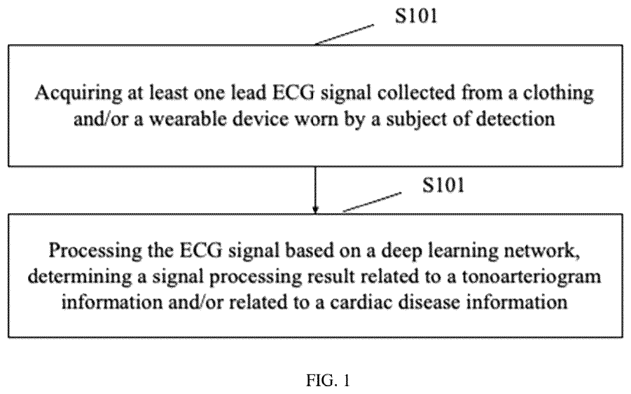

Specifically, as shown in FIG. 1, the method comprises the following steps S101-step S102:

Steps S101: acquiring at least one lead ECG signal collected from a clothing and/or a wearable device worn by a subject of detection;
Steps S102: processing the ECG signal(s) based on a deep learning network, determining a signal processing result related to a tonoarteriogram information and/or related to a cardiac disease information.

In this embodiment, the signal acquisition can be performed through the clothing and/or wearable device worn by the test subject as shown in FIG. 3; wherein the acquired ECG signal can include at least one of the following: the ECG signal of limb I lead, the ECG signal of limb II lead, the ECG signal of limb III lead, the ECG signal of chest VI lead, the ECG signal of 12 leads, the ECG signal of 15 leads The ECG signal in lead VI, the ECG signal in lead XII, and the ECG signal in lead XV. Optionally, the ECG signals associated with the limb leads can be acquired by a wearable device with ECG electrodes designed, and the ECG signals associated with the limb leads and/or the chest leads can be acquired by clothing with ECG electrodes designed.

This embodiment uses the ECG signals acquired from different leads as input to the AI model, and after processing by a neural network, the signal processing results related to tonoarteriogram information and/or related to heart disease information can be determined.

Wherein, the said signal processing results related to tonoarteriogram information include at least one of tonoarteriogram signal, systolic blood pressure information, diastolic blood pressure information, blood pressure variability information and hypertension information; specifically, the signal processing results determined by the present application can be used to obtain tonoarteriogram signals (TAG), i.e., a continuous arterial blood pressure signal, from which it is easy to obtain beat-to-beat systolic blood pressure (SBP), beat-to-beat diastolic blood pressure (DBP), and blood pressure variability (BPV) for clinical use, wherein, said signal processing results related to cardiac disease information include at least one of ECG, arrhythmia detection results, and myocardial infarction detection results. Specifically, the signal processing results identified in the present application can be used to screen for and detect different heart diseases, including but not limited to arrhythmia, atrial fibrillation, and myocardial infarction.

The following description is provided with respect to the processing of the ECG signals obtained in embodiments of the present application.

In this application embodiment, the wearable device is not only a hardware device, but it can achieve powerful functions through software support and data interaction, cloud interaction, etc. Specifically, the wearable device can be a smart watch, a smart wristband, etc. The following embodiment is illustrated with the wearable device being a smart watch as an example. Among them, the wearable device is integrated with ECG electrodes.

In a possible embodiment, the step S101 of acquiring the ECG signal of at least one lead acquired through the clothing and/or wearable device worn by the detection subject comprises at least one of the following steps A1-step A6.

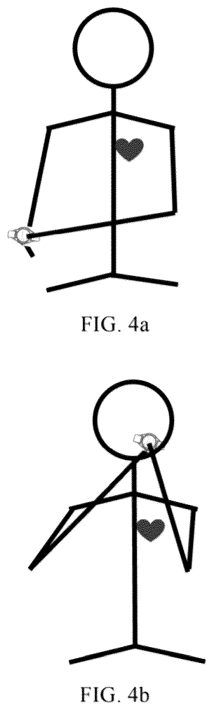

Step A1: acquiring a limb I lead ECG signal acquired while the test subject is wearing the wearable device on the right hand and the ECG electrode of the wearable device is in contact with the left hand. Specifically, as shown in FIG. 4a, a smartwatch with integrated ECG electrodes can be worn on the right hand of the test subject, and the left hand is in contact with the ECG electrodes of the smartwatch, and the ECG signal in the limb lead I can be acquired; at this time, the recorded potential difference between the left arm (LA is positive) and the right arm (RA is negative).

Step A2: acquire the Limb II lead ECG signal acquired when the test subject wears the wearable device on the right hand and the wearable device is in contact with the left side of the neck or above the left side of the neck. Specifically, a smartwatch with integrated ECG electrodes can be worn on the right hand of the test subject and the smartwatch is contacted with the left side of the neck or the area above the left side of the neck to obtain the limb II lead ECG signal; at this time, the recorded potential difference between the left leg (LL is positive) and the right arm (RA is negative).

Step A3: obtain the I, II or III lead ECG signal acquired when the test subject wears the wearable device on the right hand and the ECG electrode of the wearable device is in contact with the left hand and in contact with the left side of the neck or the area above the left side of the neck. Specifically, as shown in FIG. 4b, the smartwatch with integrated ECG electrodes can be worn on the right hand of the detection subject, followed by contacting the smartwatch with the left side of the neck or the area above the left side of the neck and contacting the left hand with the ECG electrodes of the smartwatch to acquire the I, II or III lead ECG signals of the limb.

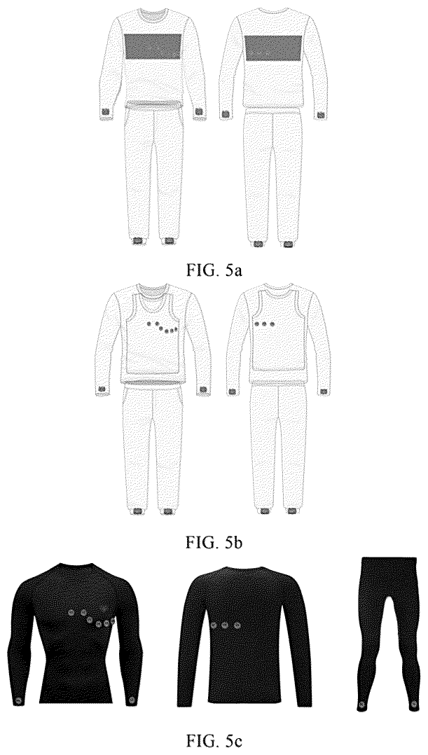

Step A4: acquiring the ECG signal of at least one of the twelve leads acquired through the first clothing worn by the detection subject; said first clothing includes ECG electrodes separately laid at the chest, wrist and ankle. Specifically, the ECG electrodes can be placed in the first clothing made of separated tights or vest with e-textile material at the chest, wrist and ankle, or in the first clothing modified based on sport tight band or vest, as shown in the left side of FIG. 5a, FIG. 5b and FIG. 5c, based on the placement of clinical twelve-lead ECG electrodes.

Step A5: obtaining the ECG signal of at least one of the fifteen leads acquired through the second clothing worn by the test subject; the said second clothing comprising ECG electrodes integrated separately at the chest, back, wrist and ankle. Specifically, as shown in the FIG. 5a, FIG. 5b, and the FIG. 5c, ECG electrodes can be placed in a second clothing made of a separated tight band or vest with e-textile material for the chest, back, wrist, and ankle, or a second clothing modified based on an athletic tight or vest, depending on the placement of clinical fifteen-lead ECG electrodes. The difference from the first clothing shown in step A4 is the addition of the placement of back ECG electrodes.

Step A6: obtaining the ECG signal of the VI lead of the chest collected through the third clothing worn by the detection subject; said third clothing includes ECG electrodes designed on the chest. Specifically, as shown in FIG. 3, the ECG electrodes may be placed in the third clothing based on the placement of the clinical chest VI lead ECG electrodes; wherein the third clothing may be made of a separated tight band or vest with an electronic fabric material or may be modified based on an athletic tight or vest.

In some examples, limb lead ECG signals and chest lead ECG signals can be obtained from clothing in conjunction with a wearable device. For example, the test subject can wear a third clothing with the wearable device to obtain the ECG signal in the limb II lead and the chest VI lead. It can be understood that the embodiment of the present application can achieve continuous monitoring of the signal 24 hours a day by integrating the ECG electrodes into the clothing, wearable device. Preferably, the clothing with ECG electrodes in the present application embodiment can be used as a smart shirt to output the collected ECG signal to an external device. The relevant design of the clothing provided in this application embodiment facilitates the reusability and comfort of the clothing.

Wherein said ECG electrodes include at least one of dry electrodes, wet electrodes, flexible electrodes, hydrogel ion electrodes, electronic fabric electrodes and non-contact electronics.

The specific process of signal processing in the embodiments of the present application is described below.

In a possible embodiment, step S102 of processing the ECG signal based on the deep learning network, determining the signal processing result related to the tonoarteriogram information and/or related to the cardiac disease information comprises, comprises steps B1-step B2.

Step B1: acquiring a low-frequency signal related to blood pressure from the ECG signal.

Step B2: processing the ECG signal and the low-frequency signal by the deep learning network, determining the signal processing result related to the tonoarteriogram information and/or related to the cardiac disease information.

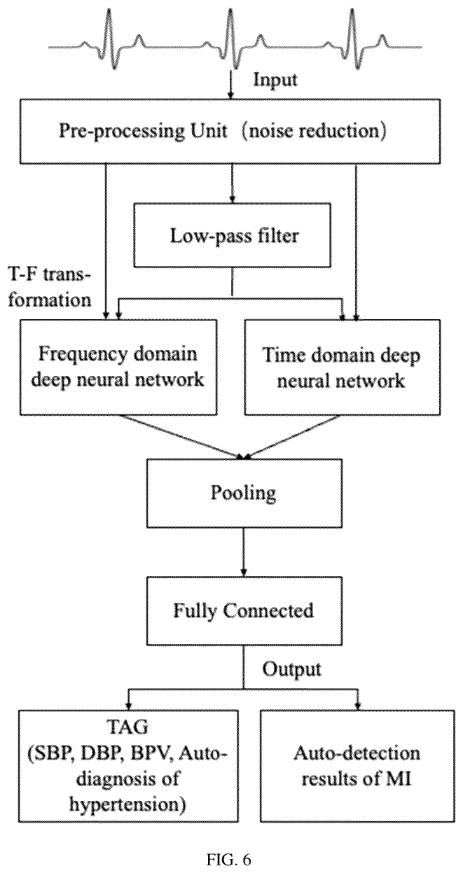

Specifically, as shown in FIG. 6, the present application embodiment can obtain the low-frequency signal related to blood pressure from the ECG signals obtained in step S101 through a low-pass filter; and then the obtained ECG signals together with the low frequency signal is used as an input to the deep learning network. That is, in this application embodiment, as shown in FIG. 4, the acquired ECG signals can be first input to the low-pass filter to obtain the low-frequency signal related to blood pressure, and then the ECG signals and the low-frequency signal can be input to the AI unit as two independent input data.

In a possible embodiment, step B2 of processing said ECG signals and said low-frequency signal by a deep learning network to determine signal processing results related to tonoarteriogram information and/or related to heart disease information comprises the following steps B21-step B23.

Step B21: obtaining frequency domain feature information by feature extraction of the said ECG signals and the said low frequency signal via a frequency domain based deep neural network, and obtaining time domain feature information by feature extraction of the said ECG signals and the said low frequency signal via a time domain based deep neural network. Specifically, as shown in FIG. 6, the inputs of the frequency domain-based deep neural network and the time-domain-based deep neural network are the same, and when performing signal processing, the two neural networks can perform parallel processing for the input data to obtain frequency domain feature information and time domain feature information, respectively.

Step B22: a pooling operation is performed on the said frequency domain feature information and the said time domain feature information to obtain the pooled feature information. Specifically, as shown in FIG. 6, the frequency domain feature information and the time domain feature information are input together to the pooling layer for the pooling operation to obtain the pooled feature information.

Step B23: the feature fusion classification process is performed on the pooled feature information via the fully connected network to determine the signal processing results related to the tonoarteriogram information and/or related to the heart disease information. Specifically, as shown in FIG. 6, the pooled feature information is input to the fully connected layer for feature fusion classification processing to finally obtain the output of the AI model (signal processing results related to the tonoarteriogram, signal processing results related to the heart disease).

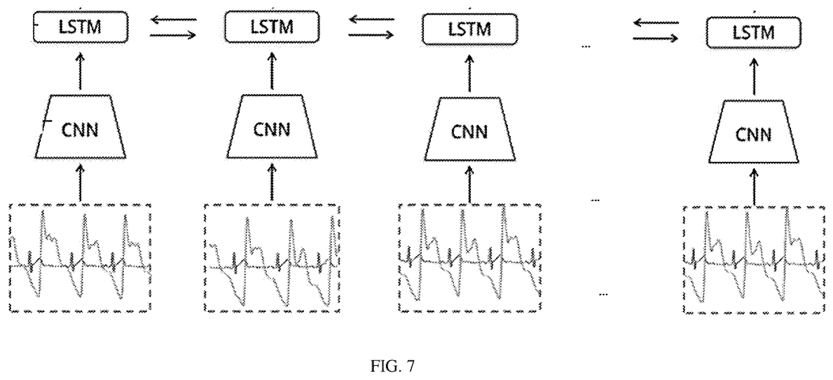

In a possible embodiment, as shown in FIG. 7, the said time-domain-based deep neural network includes sequentially connected convolutional neural network and at least one layer of long short-term memory neural network; step B21 in which the time-domain feature information is obtained by feature extraction of said ECG signal and said low-frequency signal through the time-domain-based deep neural network, comprises steps B211-step B212.

Step B211: feature extraction of the said ECG signal and the said low frequency signal by the said convolutional neural network to obtain convolutional feature information.

Step B212: processing the said convolutional feature information by the said long and short-term memory neural network to obtain time-domain feature information; the said time-domain feature information includes information on the dependence of blood pressure dynamics on time.

Specifically, as shown in FIG. 7, shown from left to right can be a plurality of consecutive ECG signals and low-frequency signals, and this application embodiment can first perform feature extraction on the ECG signals and low-frequency signals through a convolutional neural network (CNN) to obtain convolutional feature information; subsequently, the convolutional feature information can be processed through a long short-term memory neural network (LSTM,) to obtain time-domain feature information. As shown in FIG. 7, the LSTM processes the convolutional feature information input from the CNN of the previous layer, and at the same time combines the processing results of the LSTM of the same layer for signal processing, so that the output time-domain feature information can include the time-dependent information of blood pressure dynamics.

Wherein, the parameters πₜ^{k},μₜ^{k},σₜ^{k} associated with the parameters of LSTM corresponding to the current time t, respectively, and the setting of each parameter can be determined with reference to related technology, which is not limited in this application.

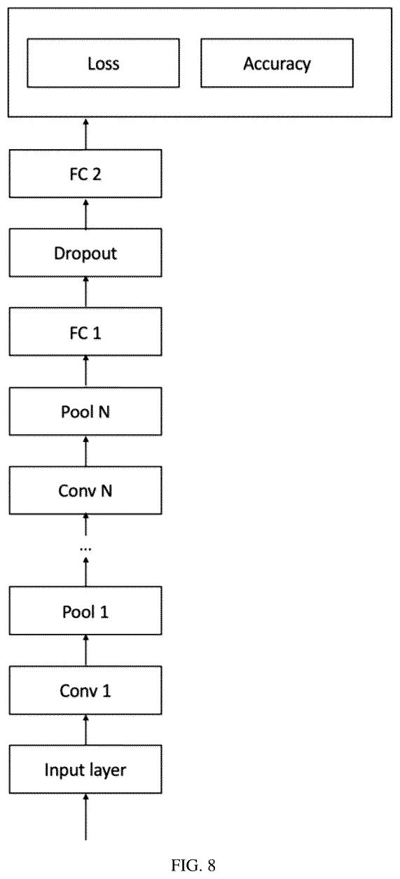

In a possible embodiment, as shown in FIG. 8, the said time-domain-based deep neural network is a time-domain-based interpretable deep learning network; the step of constructing the network comprises the following steps C1-step C6.

Step C1: reconstructing the input ECG signals with the low frequency signal through the input layer. Specifically, the reconstructing of the input can be achieved through the input layer; e.g., changing the dimension without changing the data. During the training process, the input ECG signals can be the ECG signals obtained in the embodiment (the embodiment can have the real classification result of this ECG signals), and the input low-frequency signal can be extracted from this ECG signals.

Step C2: extracting the feature information in the reconstructed ECG signals and low-frequency signal through the convolutional layer. Specifically, a convolutional layer is created to extract the input feature information.

Step C3: the pooling layer is used to pool the convolved feature information. Specifically, the pooling layer is set to reduce the amount of input data, which can be achieved by extracting the maximum value of the submatrix.

Step C4: the feature information pooled by the previous pooling layer is processed sequentially by adding any number of convolutional layers and pooling layers. Specifically, as shown in FIG. 8, after the execution of steps C2-C3, any number of N convolutional and pooling layers can be added to process the output of the previous pooling layer. where N is greater than or equal to 0.

Step C5: Classification of the feature information pooled by the last pooling layer is performed by a fully connected layer, which is set with regularization parameters. Specifically, as shown in FIG. 8, a regularization layer is set between the full connection layer 1 and the full connection layer 2, and the regularization process is designed to prevent overfitting, and the regularization parameter dropout can be set.

Step C6: Calculate the deviation of the classification results and evaluate the accuracy of the current network. Specifically, during the training of the model, the loss function (Loss) is required to calculate the loss value of the data classification and perform the optimization calculation to achieve the training of the model. Wherein, accuracy is used to characterize the performance index of the model in the evaluation mode.

Specifically, the model can be iterated based on steps C1-C6 above, and at the end of the iteration, an interpretable deep learning network based on the time domain is formed for processing ECG signals and low frequency signals. In particular, after the model is trained, the next layer of the fully connected layer 2 is the logic layer, and the logic layer can obtain the output of the fully connected layer to generate the final predicted values.

Preferably, in this embodiment, considering that the noise present in the acquired ECG signals may distort the signal and affect the accuracy of the signal processing results, as shown in FIG. 6, before performing the above step S102, filtering is also performed for the ECG signal acquired in step S101 (noise reduction is performed for the input ECG signal by the pre-processing unit). Specifically, the noise in the ECG signal is filtered to obtain a noise reduction signal before extracting the low-frequency signal, and the noise reduction signal is used as an input to the low-pass filter and the AI model.

In a possible embodiment, the deep learning-based wearable electro-tonoarteriography (ETAG) processing method for the estimation of continuous arterial blood pressure provided by embodiments of the present application further comprises at least one of the following steps D1-step D2:

Step D1: transmitting the said signal processing results to a user device to present the said processing results to the user; said user device comprising at least one of a cell phone, a watch, and glasses.
Step D2: uploading the said signal processing results to a cloud database and/or a medical platform.

Specifically, the ECG signals acquired by the wearable device and the smart shirt (signals acquired in step S101) can be communicated with at least one external electronic device via a wireless unit, and these ECG signals can be transmitted to electronic devices (e.g., cell phones, tablets, computers, and other devices) for further processing and display. In addition, the signal processing results (results obtained in step S102) may be transmitted to an external user device, such as a watch, glasses, cell phone, etc., to display the signal processing results to the user; or uploaded to a cloud database and/or a telemedicine platform to continuously record the signal processing results (e.g., tonoarteriogram information, heart disease information, etc.) determined by the test subject based on the acquired ECG signals.

In an embodiment of the present application, as shown in FIG. 3, there is also provided a system for a deep learning-based electro-tonoarteriography for the estimation of continuous arterial blood pressure, comprising an electronic apparatus and a clothing and/or a wearable apparatus for a subject of detection; the said clothing and/or the wearable device collects at least one lead ECG signal of the said subject of detection, and transmits the collected ECG signal to the said electronic apparatus via a wireless unit; wherein, the design of the clothing and wearable device can be referred to the relevant descriptions of steps A1-step A6 in the above embodiments and will not be described herein. The said electronic device performs the steps related to the deep learning-based wearable electro-tonoarteriography (ETAG) processing method for the estimation of continuous arterial blood pressure provided in the above embodiments.

Preferably, as shown in FIG. 3, the ETAG device is also equipped with an analog front-end (AFE) unit and a processing control unit connected to the analog front-end unit via Analog to Digital Converter (ADC); the processing control unit includes an MCU and a wireless unit; the ETAG device can be communicatively connected to the wireless unit in the electronic device via the wireless unit and transmit the acquired signals to the electronic device.

Preferably, the ETAG device is equipped with an analog front-end (AFE) unit and an MCU connected to the analog front-end unit via ADC, and then the MCU is connected to the wireless unit and the power supply unit, respectively; the ETAG device can transmit the collected signals to the electronic device via the wireless unit.

Preferably, as shown in FIG. 3, the electronic device is equipped with a wireless unit, a low-pass filter, an AI model and a display unit; wherein the output of the AI model can be used as the input of the display unit to realize the display of the signal processing results in the electronic device.

It should be noted that in the optional embodiment of this application, the data involved (such as ECG signal, tonoarteriogram signal) and other related data, when the above embodiment of this application is applied to a specific product or technology, it is necessary to obtain permission or consent from the object of use, and the collection, use and processing of the relevant data need to comply with the relevant laws, regulations and standards of the relevant countries and regions. In other words, if data related to the object is involved in this application embodiment, such data needs to be obtained with the authorization and consent of the object and in compliance with the relevant laws, regulations and standards of the country and region.

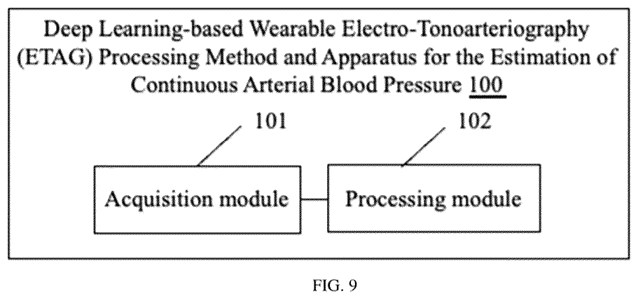

The present application embodiment provides a deep learning-based wearable electro-tonoarteriography (ETAG) apparatus for the estimation of continuous arterial blood pressure, as shown in FIG. 9, and the deep learning-based wearable electro-tonoarteriography (ETAG) apparatus for the estimation of continuous arterial blood pressure may include: an acquisition unit 101, a processing unit 102.

Wherein the acquisition unit 101 for acquiring at least one lead ECG signal collected from a clothing and/or a wearable device worn by a subject of detection; the processing unit 102 for processing the ECG signal based on a deep learning network, determining a signal processing result related to a tonoarteriogram information and/or related to a cardiac disease information.

In a possible embodiment, the wearable device is integrated with ECG electrodes; the acquisition module 101 acquiring the at least one lead ECG signal collected from a clothing and/or a wearable device worn by a subject of detection comprises at least one of the following:

acquiring a lead ECG signal I of a limb collected when the wearable device is worn at a right hand and the ECG electrode of the wearable device is in contact with a left hand of the subject of detection; acquiring a lead ECG signal II of a limb collected when the wearable device is worn at a right hand and the wearable device is in contact with a left side of a neck or a region above the left side of the neck of the subject of detection; acquiring a lead ECG signal I, II or III collected when the wearable device worn at a right hand and the ECG electrode of the wearable device is in contact with a left hand and a left side of a neck, or a region above the left side of the neck of the subject of detection; acquiring at least one lead from twelve-lead ECG signals collected from a first clothing worn by the subject of detection; the first clothing comprises the ECG electrode separately arranged at a chest portion, a wrist and an ankle; acquiring at least one lead from a fifteen-lead ECG signals collected from a second clothing worn by the subject of detection; the second clothing comprises the ECG electrode separately arranged on a chest portion, a back, a wrist and an ankle; acquiring a lead ECG signal VI of a chest portion collected from a third clothing worn by the subject of detection; the third clothing comprises an ECG electrode arranged at the chest portion.

In a possible embodiment, the ECG electrode comprises at least one of a dry electrode, a wet electrode, a flexible electrode, a hydrogel ion electrode, an electronic fabric electrode and contactless electronics.

In a possible embodiment, the processing unit 102 processes the ECG signal based on the deep learning network, determining the signal processing result related to the tonoarteriogram information and/or related to the cardiac disease information comprises: acquiring a low-frequency signal related to blood pressure from the ECG signal; processing the ECG signal and the low-frequency signal by the deep learning network, determining the signal processing result related to the tonoarteriogram information and/or related to the cardiac disease information.

In a possible embodiment, the processing unit 102 processes the ECG signal and the low-frequency signal by the deep learning network, determining the signal processing result related to the tonoarteriogram information and/or related to the cardiac disease information comprises:

performing a feature extraction on the ECG signal and the low-frequency signal by a frequency-domain based deep neural network to obtain a frequency domain feature information, and performing a feature extraction on the ECG signal and the low-frequency signal by a time-domain based deep neural network to obtain a time domain feature information; performing a pooling operation on the frequency domain feature information and the time domain feature information to obtain a pooled feature information; performing a feature fusion classification processing against the pooled feature information by a fully connected network, determining the signal processing result related to the tonoarteriogram information and/or related to the cardiac disease information.

In a possible embodiment, wherein the time-domain based deep neural network comprises a sequentially connected convolutional neural network and at least one layer of long short-term memory neural network.

The processing unit 102 performs the feature extraction on the ECG signal and the low-frequency signal by the time-domain based deep neural network to obtain the time domain feature information comprises: performing the feature extraction on the ECG signal and the low-frequency signal by the convolutional neural network to obtain a convolutional feature information; processing the convolutional feature information by the long short-term memory neural network to obtain the time domain feature information; the time domain feature information comprises dependent information of blood pressure dynamic to time.

In a possible embodiment, wherein the time-domain based deep neural network is a time-domain based interpretable deep learning network; steps of constructing the network comprises: re-structuring the input ECG signal and the low-frequency signal by an input layer; extracting a re-structured feature information of the ECG signal and the low-frequency signal by a convolutional layer; performing a pooling process on the feature information obtained from convolution by a pool layer; processing, sequentially, the feature information obtained from pooling by a previous pooling layer by adding any number of the convolutional layer and the pooling layer; performing, by a fully connected layer, a classification on the feature information obtained from the pooling by a last pooling layer, the fully connected layer is provided with a regularization parameter; calculating a deviation of a result of the classification and evaluating accuracy of a current network.

In a possible embodiment, wherein the signal processing result related to the tonoarteriogram information comprises at least one of a tonoarteriogram signal, a systolic blood pressure information, a diastolic blood pressure information, a blood pressure variation information and a high blood pressure information; the signal processing result related to the cardiac disease information comprises at least one of an electrocardiogram, an arrhythmia test result and a myocardial infarction test result.

In a possible embodiment, further comprising at least one of the following: transmitting the signal processing result to a user apparatus to display the signal processing result to a user; the user apparatus comprises at least one of a mobile phone, a watch and glasses; uploading the signal processing result to a cloud database and/or a medical platform.

The device of the present application embodiment can perform the method provided in the present application embodiment with similar implementation principles. The actions performed by the modules in the device of the present application embodiments are corresponding to the steps in the method of the present application embodiments, and the detailed functional description of the modules of the device can be specifically referred to the description in the corresponding method shown in the previous section, which will not be repeated here.

The processing results, ECG signals, etc. involved in the embodiments of the present application can be stored by blockchain technology. The blockchain referred to in this application is a new application model of computer technology such as distributed data storage, peer-to-peer transmission, consensus mechanism, and encryption algorithm. A blockchain, essentially a decentralized database, is a string of data blocks generated using cryptographic methods associated with each block containing a certain amount of processed data for verifying the validity of its information (forgery-proof) and generating the next block. Blockchain can include a blockchain underlying platform, a platform product service layer, and an application service layer

Provided in this application embodiment is an electronic device comprising a memory, a processor and a computer program stored on the memory, the processor executing the steps of the above computer program to implement a deep learning-based wearable electro-tonoarteriography (ETAG) processing method for the estimation of continuous arterial blood pressure, which can be implemented compared to related technologies: specifically, the acquisition of ECG signals can be performed by clothing and/or wearable devices worn by the test subject. Since the clothing and the wearable device are very easily wearable by the test subject based on the testing needs, continuous signal acquisition can be performed, from which more physiologically meaningful signals can be obtained for processing, and the implementation of this operation facilitates the acquisition of continuous arterial blood pressure signals and/or automatic diagnosis of heart diseases; on this basis, after acquiring the ECG signal in at least one lead, the ECG signal can be processed based on deep learning network to process this ECG signal and determine the signal processing results related to tonoarteriogram information and/or heart disease information; the implementation of this operation can directly perform the estimation of tonoarteriogram signal and heart disease based on the acquired ECG signals without combining the ECG signal with the photoplethysmographic signal, which is conducive to reducing the cost of device fabrication and improving the accuracy of signal processing results.

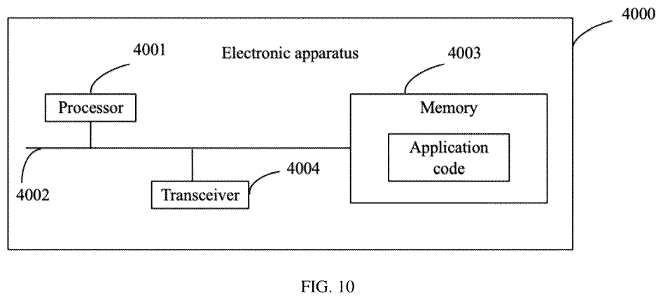

In an optional embodiment an electronic apparatus is provided, as shown in FIG. 10, wherein the electronic apparatus 4000 shown in FIG. 10 includes: a processor 4001 and a memory 4003. wherein the processor 4001 and the memory 4003 are connected, e.g., via a bus 4002. Optionally, the electronic apparatus 4000 may also include a transceiver 4004, which may be used for data interaction between this electronic device and other electronic devices, such as the sending of data and/or the receiving of data, etc. It should be noted that the transceiver 4004 is not limited to one in practical applications, and the structure of the electronic apparatus 4000 does not constitute a limitation of this application embodiment.

The processor 4001 may be a CPU (Central Processing Unit), a general-purpose processor, a DSP (Digital Signal Processor), an ASIC (Application Specific Integrated Circuit), an FPGA (Field Programmable Gate Array) or other programmable logic device, transistorized logic device, hardware component, or any combination thereof. It may implement or execute various exemplary logic boxes, modules, and circuits described in conjunction with the disclosure of this application. Processor 4001 may also be a combination that implements a computing function, such as a combination containing one or more microprocessors, a combination of a DSP and a microprocessor, etc.

Bus 4002 may include a pathway to transfer information between the above components. Bus 4002 may be a PCI (Peripheral Component Interconnect) bus or an EISA (Extended Industry Standard Architecture) bus, etc. Bus 4002 can be divided into address bus, data bus, control bus, etc. For the convenience of representation, only one thick line is used in FIG. 10, but it does not mean that there is only one bus or one type of bus.

Memory 4003 can be ROM (Read Only Memory) or other types of static storage devices that can store static information and instructions, RAM (Random Access Memory) or other types of dynamic storage devices that can store information and instructions, or EEPROM (Electrically Erasable Programmable Read Only Memory), CD-ROM (Compact Disc Read Only Memory) or other optical disc storage, optical disc storage or other optical disc storage, disk storage media, other magnetic storage devices, or any other media capable of being used to carry or store computer programs and capable of being read by a computer, without limitation herein.

Memory 4003 is used to store a computer program for executing an embodiment of the present application and is controlled for execution by processor 4001. The processor 4001 is used to execute the computer program stored in the memory 4003 to implement the steps shown in the preceding method embodiment.

Wherein, the electronic device includes, but is not limited to a server, a terminal.

Embodiments of the present application provide a computer-readable storage medium on which a computer program is stored, wherein, when the computer program is executed by a processor, implements the steps and corresponding contents of the foregoing method embodiments.

Embodiments of the present application also provide a computer program product comprising a computer program, wherein, when the computer program is executed by a processor, implements the steps and corresponding contents of the foregoing method embodiments.

The terms “first,” “second,” “third,” “fourth” in the specification and claims of this application and in the accompanying drawings above”, “1”, “2”, etc. (if present) are used to distinguish similar objects and need not be used to describe a particular order or sequence. It should be understood that the data so used is interchangeable where appropriate so that embodiments of the present application described herein can be implemented in an order other than that illustrated or described in the text.

It should be understood that while the flow diagrams of embodiments of the present application indicate the individual operational steps by arrows, the order in which these steps are performed is not limited to the order indicated by the arrows. Unless explicitly stated herein, in some implementation scenarios of embodiments of the present application, the implementation steps in the respective flowcharts may be performed in other orders as desired. In addition, some or all of the steps in each flowchart may include multiple sub-steps or multiple stages based on actual implementation scenarios. Some or all of these sub-steps or phases may be executed at the same moment, and each of these sub-steps or phases may also be executed separately at different moments. In scenarios where the execution time is different, the order of execution of these sub-steps or stages can be flexibly configured according to the needs, and this application embodiment is not limited in this regard.

It should be noted that for a person of ordinary skill in the art, other similar means of implementation based on the technical idea of the present application, without departing from the technical idea of the present application, also fall within the scope of protection of the embodiments of the present application.

We claim:

1. A deep learning-based wearable electro-tonoarteriography (ETAG) processing method for the estimation of continuous arterial blood pressure, comprising:
acquiring at least one lead electrocardiogram (ECG) signal collected from a clothing and/or a wearable device worn by a subject of detection;
processing the ECG signal based on a deep learning network, determining a signal processing result related to a tonoarteriogram information and/or related to a cardiac disease information.

2. The method according to claim 1, wherein the wearable device is integrated with an ECG electrode; the step of acquiring the at least one lead ECG signal collected from a clothing and/or a wearable device worn by a subject of detection comprises at least one of the following:
acquiring a lead ECG signal I of a limb collected when the wearable device is worn at a right hand and the ECG electrode of the wearable device is in contact with a left hand of the subject of detection;
acquiring a lead ECG signal II of a limb collected when the wearable device is worn at a right hand and the wearable device is in contact with a left side of a neck or a region above the left side of the neck of the subject of detection;
acquiring a lead ECG signal I, II or III collected when the wearable device worn at a right hand and the ECG electrode of the wearable device is in contact with a left hand and a left side of a neck, or a region above the left side of the neck of the subject of detection;
acquiring at least one lead from twelve-lead ECG signals collected from a first clothing worn by the subject of detection; the first clothing comprises the ECG electrode separately arranged at a chest portion, a wrist and an ankle;
acquiring at least one lead from a fifteen-lead ECG signals collected from a second clothing worn by the subject of detection; the second clothing comprises the ECG electrode separately arranged on a chest portion, a back, a wrist and an ankle;
acquiring a lead ECG signal VI of a chest portion collected from a third clothing worn by the subject of detection; the third clothing comprises an ECG electrode arranged at the chest portion;

3. The method according to claim 2, wherein the ECG electrode comprises at least one of a dry electrode, a wet electrode, a flexible electrode, a hydrogel ion electrode, an electronic fabric electrode and contactless electronics.

4. The method according to claim 1, wherein the step of processing the ECG signal based on the deep learning network, determining the signal processing result related to the tonoarteriogram information and/or related to the cardiac disease information comprises:
acquiring a low-frequency signal related to blood pressure from the ECG signal;
processing the ECG signal and the low-frequency signal by the deep learning network, determining the signal processing result related to the tonoarteriogram information and/or related to the cardiac disease information.

5. The method according to claim 4, wherein the step of processing the ECG signal and the low-frequency signal by the deep learning network, determining the signal processing result related to the tonoarteriogram information and/or related to the cardiac disease information comprises:
performing a feature extraction on the ECG signal and the low-frequency signal by a frequency-domain based deep neural network to obtain a frequency domain feature information, and performing a feature extraction on the ECG signal and the low-frequency signal by a time-domain based deep neural network to obtain a time domain feature information;
performing a pooling operation on the frequency domain feature information and the time domain feature information to obtain a pooled feature information;
performing a feature fusion classification processing against the pooled feature information by a fully connected network, determining the signal processing result related to the tonoarteriogram information and/or related to the cardiac disease information.

6. The method according to claim 5, wherein the time-domain based deep neural network comprises a sequentially connected convolutional neural network and at least one layer of long short-term memory neural network;
the step of performing the feature extraction on the ECG signal and the low-frequency signal by the time-domain based deep neural network to obtain the time domain feature information comprises:
performing the feature extraction on the ECG signal and the low-frequency signal by the convolutional neural network to obtain a convolutional feature information;
processing the convolutional feature information by the long short-term memory neural network to obtain the time domain feature information; the time domain feature information comprises dependent information of blood pressure dynamic to time.

7. The method according to claim 5, wherein the time-domain based deep neural network is a time-domain based interpretable deep learning network; steps of constructing the network comprises:
re-structuring the input ECG signal and the low-frequency signal by an input layer;
extracting a re-structured feature information of the ECG signal and the low-frequency signal by a convolutional layer;
performing a pooling process on the feature information obtained from convolution by a pool layer;
processing, sequentially, the feature information obtained from pooling by a previous pooling layer by adding any number of the convolutional layer and the pooling layer;
performing, by a fully connected layer, a classification on the feature information obtained from the pooling by a last pooling layer, the fully connected layer is provided with a regularization parameter;
calculating a deviation of a result of the classification and evaluating accuracy of a current network.

8. The method according to claim 1, wherein the signal processing result related to the tonoarteriogram information comprises at least one of a tonoarteriogram signal, a systolic blood pressure information, a diastolic blood pressure information, a blood pressure variation information and a high blood pressure information;
the signal processing result related to the cardiac disease information comprises at least one of an electrocardiogram, an arrhythmia test result and a myocardial infarction test result.

9. The method according to claim 1, further comprising at least one of the following:
transmitting the signal processing result to a user apparatus to display the signal processing result to a user; the user apparatus comprises at least one of a mobile phone, a watch and glasses;
uploading the signal processing result to a cloud database and/or a medical platform.

10. A deep learning-based wearable electro-tonoarteriography (ETAG) apparatus for the estimation of continuous arterial blood pressure, comprising:
an acquiring unit for acquiring at least one lead ECG signal collected from a clothing and/or a wearable device worn by a subject of detection;
a processing unit for processing the ECG signal based on a deep learning network, determining a signal processing result related to a tonoarteriogram information and/or related to a cardiac disease information.

11. A system for a deep learning-based electro-tonoarteriography for the estimation of continuous arterial blood pressure, comprising an electronic apparatus and a clothing and/or a wearable apparatus for a subject of detection; the clothing and/or the wearable device collects at least one lead ECG signal of the subject of detection, and transmits the collected ECG signal to the electronic apparatus via a wireless unit; the electronic apparatus implements the method steps of claim 1.
